(19) United States
(12) Patent Application Publication
(10) Pub. No.: US 2025/0262141 A1
(43) Pub. Date: Aug. 21, 2025

(54) VAGINA SKINCARE MASK

(71) Applicant: Jennifer Erica Garcia, Aliso Viejo, CA (US)

(72) Inventor: Jennifer Erica Garcia, Aliso Viejo, CA (US)

(21) Appl. No.: 18/444,726

(22) Filed: Feb. 18, 2024

(51) Int. Cl. A61K 8/65; A61K 8/02; A61K 8/34; A61K 8/73; A61K 8/92; A61K 8/9789; A61K 8/9794; A61Q 19/00

(52) U.S. Cl. CPC A61K 8/65; A61K 8/0208; A61K 8/345; A61K 8/735; A61K 8/922; A61K 8/9789; A61K 8/9794; A61Q 19/00; A61K 2800/10

(57) ABSTRACT

An embodiment of the present invention is a vaginal mask, comprising: an upper portion configured to attach along the waistline a female; and a lower portion configured to attach between the legs narrowing as the lower portion extends downward from the upper portion, wherein: the upper and lower portions comprise an outer layer and an inner fluid layer. In a preferred embodiment, the vaginal mask contains hydrolyzed collagen.

### BACKGROUND

Skin care face masks have been described in the prior art patent literature.

For example, WO2011/004834 discloses a skin care face mask comprising a liquid-retaining layer and an adhesion layer; the liquid-retaining layer comprises a first nonwoven structural member comprising a first fiber and has an ability to absorb a liquid component, and the adhesion layer is contactable with a skin, is formed on at least one side of the liquid-retaining layer and is permeable to the liquid component, wherein the adhesion layer comprises a second nonwoven structural member comprising a second fiber.

U.S. Pat. No. 10,743,638 discloses a beauty care pack including a release film and a beauty care sheet which is separably attached to the release film, wherein the beauty care sheet comprises: a support; a fiber layer which is laminated on the support and in which water-soluble polymers are formed in the form of a nanofiber web; a moisturizing layer which is laminated on the fiber layer and consists of the water-soluble polymers and moisturizing materials; and a beauty care layer which is laminated on the moisturizing layer and in which the water-soluble polymers and functional materials are formed in the form of a nanofiber web, such that active ingredients are absorbed into the skin while being dissolved by moisture.

Such skin care face masks allow the skin to be maintained in a moist state. A skin care mask is typically formed from a sheet impregnated with one or more skin care compositions and has a function of transferring the composition to the skin. In order to transfer the composition to the skin, the sheet requires closely adhering to the skin. Thus, for the purpose of successfully allowing the sheet of the facial mask to have contact with the skin, the shape of the sheet has great importance.

It has not been taught or suggested in the prior art that a skin care mask be applied to skin in the area of the female vagina, however.

### SUMMARY OF INVENTION

Therefore, the present invention provides a vaginal mask. The vaginal mask can be a collagen mask. The mask moisturizes and soothes the vaginal area providing relief from redness and bumps while protecting against ingrown hairs. The mask moisturizes while restoring the vaginal skin's barrier to soften the look and feel of the skin in the female vagina area.

According to an aspect of the present invention, there is provided a vaginal mask, comprising: an upper portion configured to attach along the waistline a female; and a lower portion configured to attach between the legs narrowing as the lower portion extends downward from the upper portion, wherein: the upper and lower portions comprise an outer layer and an inner fluid layer.

In a preferred embodiment, the vaginal mask contains hydrolyzed collagen.

### BRIEF DESCRIPTION OF THE DRAWINGS

FIG. 1 illustrates a vaginal mask according to an embodiment of the present invention.

FIG. 2 illustrates the mask applied to the vagina area of a female.

### DETAILED DESCRIPTION

The mask of the present invention helps prevent ingrown hairs, reduce razor bumps, improve vaginal odor, and beautify the vaginal area.

Several vulvar skin conditions usually associated with other areas of the body can be improved by the mask of the present invention.

Lichen sclerosus, lichen simplex chronicus, eczema, and psoriasis are among the conditions which can be improved by the mask of the present invention.

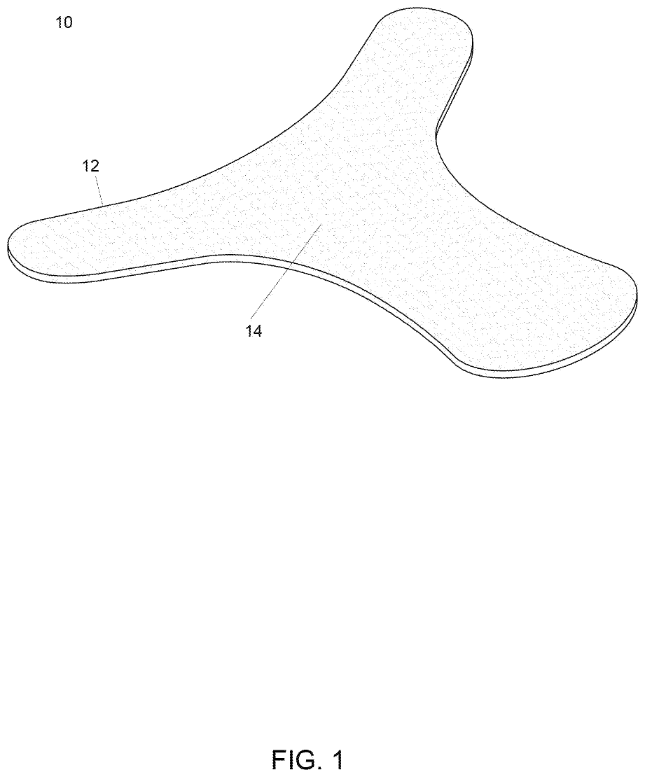

FIG. 1 illustrates a vaginal mask according to an embodiment of the present invention.

Vaginal mask 10 comprises surface 14 and interior 12. The interior 12 contains collagen in a preferred embodiment, which can be transferred to the vaginal skin through surface 14.

Interior 12 is generally comprised of fluid. The fluid can contain a variety of skincare preparations. The fluid can have a jelly-like consistency, for example.

The surface 14 can be a porous woven fabric or a nonwoven fabric, for example.

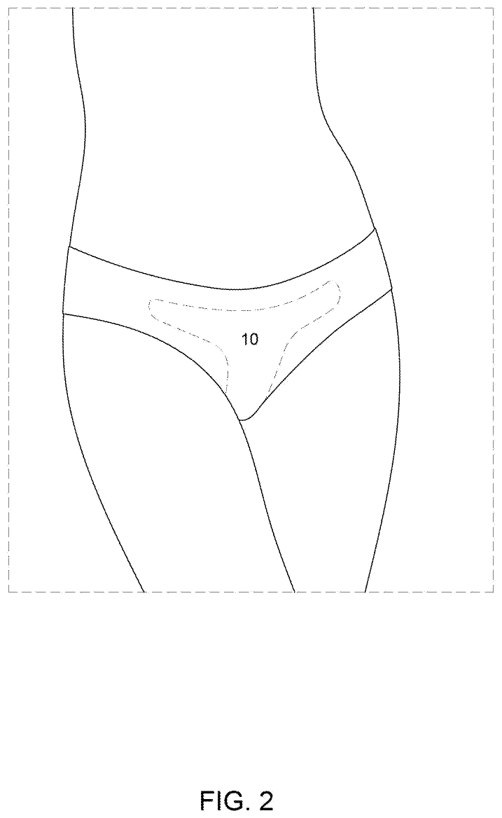

FIG. 2 illustrates the mask applied to the vagina area of a female.

Vaginal mask 10 is optimally shaped to apply to the vaginal area of a female. The mask should ideally be applied for 15-20 minutes and then removed.

A variety of scents can be applied to the mask, for example, a candy rose scent in a preferred embodiment, provided in part by rosa rugosa flower oil.

Mask ingredients can include water, glycerin, propylene glycol, betaine, chondrus crispus powder, glucomannan, trehalose, butylene glycol, hydroxyacetophenone, 1,2-hexanediol, hydrolyzed collagen, pentylene glycol, hydroxyethylcellulose, matrine, tocopheryl acetate, carbomer, triethanolamine, aloe barbadensis leaf extract, sodium hyaluronate, sea salt, mica, hydroxyphenyl propamidobenzoic acid, Bergenia crassifolia root extract, and rosa rugosa flower oil.

Hydrolyzed collagen is particularly important as an active ingredient in mask embodiments of the present invention.

Other ingredients can be added, subtracted or substituted with similar compounds without affecting the biologic activity.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A vaginal mask, comprising:
an upper portion configured to attach along the waistline a female; and
a lower portion configured to attach between the legs narrowing as the lower portion extends downward from the upper portion, wherein:
the upper and lower portions comprise an outer layer and an inner fluid layer.

2. The vaginal mask of claim 1, wherein one or more of the outer layer and the inner fluid layer contains hydrolyzed collagen.

3. The vaginal mask of claim 1, wherein one or more of the outer layer and the inner fluid layer contain a scented compound.

4. The vaginal mask of claim 3, wherein the scented compound is rosa rugosa flower oil.

5. The vaginal mask of claim 1, wherein one or more of the outer layer and the inner fluid layer contains aloe barbadensis leaf extract.

6. The vaginal mask of claim 1, wherein one or more of the outer layer and the inner fluid layer contains Bergenia crassifolia root extract.

7. The vaginal mask of claim 1, wherein one or more of the outer layer and the inner fluid layer contains sea salt.

8. The vaginal mask of claim 1, wherein one or more of the outer layer and the inner fluid layer contains glycerin.

9. The vaginal mask of claim 1, wherein one or more of the outer layer and the inner fluid layer contains sodium hyaluronate.

10. A method of applying the vaginal mask of claim 1, comprising:
placing the mask on the vaginal area for between 15 to 20 minutes; and
removing the mask.
